(19) United States
(12) Patent Application Publication
(10) Pub. No.: US 2021/0350928 A1
(43) Pub. Date: Nov. 11, 2021

(54) A SYSTEM AND METHOD FOR IMPUTING MISSING DATA IN A DATASET, A METHOD AND SYSTEM FOR DETERMINING A HEALTH CONDITION OF A PERSON, AND A METHOD AND SYSTEM OF CALCULATING AN INSURANCE PREMIUM

(71) Applicants: UNIVERSITY OF JOHANNESBURG, Johannesburg (ZA); UNIVERSITY OF THE WITWATERSRAND, JOHANNESBURG, Johannesburg (ZA)

(72) Inventors: Tshilidzi MARWALA, Johannesburg (ZA); Rendani MBUVHA, Johannesburg (ZA)

(21) Appl. No.: 17/278,153

(22) PCT Filed: Sep. 20, 2019
(86) PCT No.: PCT/IB2019/057974

§ 371 Date: Mar. 19, 2021

(30) Foreign Application Priority Data
Sep. 21, 2018 (ZA) 2018/06344

(51) Int. Cl. G16H 50/20; G06Q 40/08; G16H 50/30; G16H 10/60; G06N 3/04

(52) U.S. Cl. CPC G16H 50/20; G06Q 40/08; G06N 3/0454; G16H 10/60; G16H 50/30

(57) ABSTRACT

This invention relates to systems and methods for imputing missing data in a dataset, for determining a health condition of a person, and for calculating an insurance premium. In particular, the method described herein employs a trained autoencoder system which is configured to receive an input dataset comprising input data which has data missing therefrom. In a preferred example embodiment, the input data contains data associated with a person and the missing data is an HIV and/or Syphilis status of the person. The trained autoencoder system is configured to impute the missing data from the input dataset, which in the case of the preferred example embodiment is to impute or predict the HIV and/or Syphilis status of the person.

### FIELD OF INVENTION

THIS INVENTION relates to systems and methods for imputing missing data in a dataset, for determining a health condition of a person, and for calculating an insurance premium.

### BACKGROUND TO THE INVENTION

The Inventors have noted that in numerous computational tasks in business, science, engineering etc the problem of missing data in datasets often arises due to inter alia sensor and system failure, non-collection, and data losses.

In some cases, stop-gap measure such as the use of redundancies and mean imputation have been successful in obtaining information pertaining to the missing data.

However, the Inventors have noticed that a need exists to provide an alternative method for imputation of missing data in a dataset.

Moreover, the Inventors have noticed that for insurance underwriting purposes, it is often important to be aware of the health condition of a person to be insured based on pathological tests performed by healthcare professionals. In most instances, outcomes of pathological tests are used to determine whether to accept a potential person to be insured and if accepted the terms and conditions of such acceptance which is then codified in suitable contracts. These terms may include, inter alia, the price/premium, the claim conditions and the sum at risk.

In some cases, pathological testing may be difficult to administer, for example, if a person to be insured cannot avail themselves to a suitable testing facility. This may be the case in rural locations where it is difficult for persons to be insured to travel to suitable testing facilities.

This dilemma poses a problem for insurers in that they cannot effectively expand their service offerings to these persons.

Therefore, it is thus another object of the invention to determine a health condition of a person based on a dataset associated therewith without the need for conventional pathological testing.

### SUMMARY OF THE INVENTION

According to a first aspect of the invention, there is provided a method for a computer system to impute data missing from an input dataset, wherein the method comprises:

receiving, by a trained autoencoder system, an input dataset comprising input data which has data missing therefrom;

processing the input data with a trained autoencoder system comprising a plurality of stacked trained autoencoders, wherein the trained autoencoder system has been trained with one or more complete datasets;

generating an output dataset comprising output data from the trained autoencoder system based on the input dataset;

minimising an overall error function based on a relationship between the input dataset and the generated data output dataset from the trained autoencoder system to impute the data missing from the input dataset which preserves non-linear relationships within the autoencoder system; and

generating an output based on the imputed data missing from the input dataset.

The method may comprise generating the output substantially in real-time.

It will be appreciated that the method above as well as any method described herein may be a computer-implemented method.

The complete dataset, the input dataset, and the output dataset may each have a similar structure. The datasets may have the same dimensionality. Moreover, the datasets may have the same predetermined number of fields. It follows that the input dataset may have one or more fields with missing data whereas the complete dataset may have all the data provided in the fields. In other words, the complete dataset may have no data missing from the respective fields.

The datasets may be in the form of vectors of data. The dimensions of each vector are determined by the number of fields.

The method may comprise training an autoencoder system comprising a plurality of stacked autoencoders with one or more complete datasets to generate the trained autoencoder system comprising a plurality of trained autoencoders.

Each autoencoder may comprise a neural network. The neural network may comprise an input layer, at least one hidden layer, and an output layer. The input layer may have the same dimensionality as the output layer. In some example embodiments, each autoencoder may comprise a plurality of hidden layers. The hidden layers may have a lower dimensionality than the input and output layers.

The neural network may be formed by way of multi-layer perceptrons, radial basis functions, deep networks, and the like.

The step of training the autoencoder system may comprise, for each autoencoder in the autoencoder system:
inputting one or more complete datasets into an autoencoder;
generating an output dataset which is outputted from the autoencoder based on the complete dataset; and
deriving optimal weights for the respective autoencoder or the weighted encoder function of the autoencoder by minimising an error function associated with the autoencoder to yield a trained autoencoder.

The method may comprise training each of the plurality of autoencoders in the autoencoder system in parallel to derive the trained autoencoder system. The trained autoencoder system may therefore comprise trained autoencoders having the derived optimal weights assigned thereto.

Differently defined, each trained autoencoder may comprise a suitable weighted encoder function having derived optimal weights. The method may therefore comprise deriving the respective optimal weights for the respective weighted encoder functions of each autoencoder.

The error function of each autoencoder may be a distance metric between the complete dataset inputted to the autoencoder and the output dataset from the autoencoder. In one example embodiment, the error function of each autoencoder may be a Euclidean distance between the complete dataset inputted to the autoencoder and the output dataset from each autoencoder. The error function of each autoencoder in the autoencoder system may be a square of the difference between the complete dataset which is inputted into the autoencoder and the output dataset generated and outputted by the autoencoder.

The minimisation of the error function may be done by computational intelligence techniques such as gradient decent, Particle Swam optimisation, genetic algorithm, or the like. It will be understood by those skilled in the art that these techniques are recursive and iterative.

The step of minimising the overall error function to impute the data missing from the input dataset may comprise minimising the overall error function for the trained autoencoder system with the optimal weights associated with each autoencoder of the autoencoder system fixed.

The trained autoencoder system may be an error weighted stacked autoencoder system. The method may therefore comprise determining an output dataset from the trained autoencoder system by combining the products of the output datasets of each trained autoencoder and an error ratio associated with the respective trained autoencoders. The error ratio may be based on the error of a particular autoencoder and the overall error of the autoencoder system. The output dataset from the trained autoencoder system may be outputted from the trained autoencoder system.

The overall error function of the trained autoencoder system may be a distance metric between the input dataset inputted to the trained autoencoder system and the output dataset from the trained autoencoder system. In one example embodiment, the overall error function may be a Euclidean distance between the input dataset inputted to the trained autoencoder system and the output dataset from the trained autoencoder system. The error function may be a square of the difference between the input dataset and the output dataset from the trained autoencoder system as described above.

The minimisation of the overall error function may be done by way of computational intelligence techniques such Particle Swam optimisation, genetic algorithm, or the like. This step may be referred to as an optimisation step. It will be appreciated that this step is inherently iterative and recursive.

In one example embodiment, the complete dataset may be in the form of complete antenatal data. In addition to fields pertaining to HIV (Human Immunodeficiency Virus) status and/or Syphilis status, the fields may be selected from a group comprising race, region, age of the mother, age of the father, education level of the mother, gravidity, parity, province of origin, region of origin, and a regional weighting parameter (WTREV).

The method may comprise normalising the antenatal data to a vector format. The dimensions of input and output layers of each autoencoder in the autoencoder system may be based on the number of fields selected.

The input dataset may be missing fields pertaining to one or both of HIV and Syphilis status. The method may therefor comprise imputing one or both of HIV and Syphilis status from the input dataset.

According to a second aspect of the invention, there is provided a computer system to impute data missing from an input dataset, wherein the system comprises:

a data storage device storing data; and

one or more processors configured to:
receive an input dataset comprising input data which has data missing therefrom;
process the input data with a trained autoencoder system wherein the trained autoencoder system comprises a plurality of stacked trained autoencoders, wherein the trained autoencoder system has been trained with one or more complete datasets;
generate an output dataset comprising output data from the trained autoencoder system based on the input dataset;
minimise an overall error function based on a relationship between the input dataset and the generated data output dataset from the trained autoencoder system to impute the data missing from the input dataset which preserves non-linear relationships within the trained autoencoder system; and
generate an output based on the imputed data missing from the input dataset.

The one or more processors may be configured to provide the trained autoencoder system.

The one or more processors may be configured to train an autoencoder system comprising a plurality of stacked autoencoders with one or more complete datasets to generate the trained autoencoder system comprising a plurality of trained autoencoders;

The one or more processors may be configured to train the autoencoder system by:
inputting, to each autoencoder in the autoencoder system, one or more complete datasets;
generating an output dataset which is outputted from each autoencoder based on the complete dataset; and
deriving optimal weights for the respective autoencoder or the weighted encoder function of the autoencoder by minimising an error function associated with the autoencoder so as to yield a trained autoencoder.

The one or more processors may be configured to train each of the plurality of autoencoders in the autoencoder system in parallel to derive the trained autoencoder system. The trained autoencoder system may therefore comprise trained autoencoders having the derived optimal weights assigned thereto.

The one or more processors may be configured to minimise the error function by applying computational intelligence techniques such as gradient decent, Particle Swam optimisation, genetic algorithm, or the like.

The one or more processors may be configured to minimise the overall error function for the trained autoencoder system with the optimal weights associated with each autoencoder of the autoencoder system fixed.

The one or more processors may be configured to determine an output dataset from the trained autoencoder system by combining the products of the output datasets of each trained autoencoder and an error ratio associated with the respective trained autoencoders. The error ratio may be based on the error of a particular autoencoder and the overall error of the autoencoder system. The one or more processors may be configured to output the output dataset from the trained autoencoder system.

The overall error function may be a square of the difference between the input dataset and the output dataset from the trained autoencoder system.

The one or more processors may be configured to minimise the overall error function by way of computational intelligence techniques such as gradient decent, Particle Swam optimisation, genetic algorithm, or the like.

The system may be configured to generate the output substantially in real-time.

It will be appreciated by those skilled in the art that the comments above regarding the first aspect of the invention apply herein as well, mutatis mutandis. This is because the method described above may be implemented by the system described above.

According to a third aspect of the invention there is provided a method of determining a health condition of a person, wherein the method comprises:

receiving, by a trained autoencoder system, an input dataset comprising input data which has data missing therefrom, wherein the input data is comprises demographic data associated with the person and the data missing from the input data is one or more health conditions associated with the person;

processing the input data with a trained autoencoder system comprising a plurality of stacked trained autoencoders, wherein the trained autoencoder system has been trained with a plurality of complete datasets comprising complete data, wherein each complete dataset comprises complete data which comprises demographic data and one or more health conditions associated with a person;

generating an output dataset comprising output data from the trained autoencoder system based on the input dataset;

minimising an overall error function based on a relationship between the input dataset and the generated data output dataset from the trained autoencoder system to impute the data missing from the input dataset corresponding to the one or more health conditions associated with the person, wherein the imputed data missing from the input dataset preserves non-linear relationships within the trained autoencoder system; and

generating an output based on the imputed data missing from the input dataset corresponding to the one or more health conditions.

The health condition may be a predictive diagnosis of a malady. This may be a positive or negative prediction of the malady. In a preferred example embodiment, the health condition is a positive or negative predictive diagnosis of a person having HIV (Human Immunodeficiency Virus) and/or Syphilis based on the input dataset to the trained autoencoder system.

It follows that the input dataset may have a plurality of data fields comprising input data corresponding to demographic data pertaining to the person and input data missing in fields which correspond to HIV and/or Syphilis status. On the other hand, the complete dataset may have demographic data, as well as HIV and Syphilis status provided in the fields. In other words, the complete dataset may have no data missing from the respective fields.

The complete dataset may comprise antenatal data comprising demographic data as well as HIV status and Syphilis status information associated with a plurality of people.

The demographic data contained in the complete dataset may comprise data selected from a group comprising race, region, age of the mother, age of the father, education level of the mother, gravidity, parity, province of origin, region of origin, and a regional weighting parameter (WTREV).

From the foregoing, it will be appreciated that the input data may also comprise demographic data selected from a group comprising race, region, age of the mother, age of the father, education level of the mother, gravidity, parity, province of origin, region of origin, and a regional weighting parameter (WTREV).

The method may comprise normalising the antenatal data to a vector format for the complete dataset.

The method may comprise the prior steps of:
prompting a person for demographic data;
receiving the demographic data from the person; and
generating the input dataset for receipt by the trained autoencoder system, wherein the input dataset comprises the demographic data received from the person and has data fields pertaining to the HIV status and/or Syphilis status of the person missing.

The step of generating the input dataset may comprise normalising the received demographic data into a predetermined format required by the trained autoencoder system. The method may therefore comprise vectorising the demographic data received by the person.

The method may comprise a step of determining if the person is a female, wherein if the person is a female, the method may comprise prompting the female person for antenatal data prior to imputing the data missing from the input dataset. This step may be to allow for an input dataset to be generated which is substantially similar to the complete dataset used to train the autoencoder, albeit with missing data.

If the person is not a female, the method may comprise determining if the male person has a female partner. If the male person has a female partner, the method may comprise prompting the male person for antenatal data pertaining to their female partner prior to imputing the data missing from the input dataset.

The step of determining whether the person is male or female and/or if they have a female partner may be done by prompting the person and receiving suitable responses.

It will be understood by those skilled in the art that the method steps and remarks previously described with reference to the first aspect of the invention apply herein as well, mutatis mutandis. This is because the method according to the third aspect of the invention is an implementation/application of the method according to the first aspect of the invention.

According to a fourth aspect of the invention, there is provided a system for determining a health condition of a person, wherein the system comprises:

a memory store; and

one or more processor configured to:
receive, by a trained autoencoder system, an input dataset comprising input data which has data missing therefrom, wherein the input data is comprises demographic data associated with the person and the data missing from the input data is one or more health conditions associated with the person;
process the input data with a trained autoencoder system comprising a plurality of stacked trained autoencoders, wherein the trained autoencoder system has been trained with a plurality of complete datasets comprising complete data, wherein each complete dataset comprises complete data which comprises demographic data and one or more health conditions associated with a person;
generate an output dataset comprising output data from the trained autoencoder system based on the input dataset;
minimise an overall error function based on a relationship between the input dataset and the generated data output dataset from the trained autoencoder system to impute the data missing from the input dataset corresponding to the one or more health conditions associated with the person, wherein the imputed data missing from the input dataset preserves non-linear relationships within the trained autoencoder system; and
generate an output based on the imputed data missing from the input dataset corresponding to the one or more health conditions.

The processor may provide the trained autoencoder system.

The health condition may be a predictive diagnosis of a malady. This may be a positive or negative prediction of the malady. In a preferred example embodiment, the health condition is a positive or negative predictive diagnosis of a person having HIV (Human Immunodeficiency Virus) and/or Syphilis based on the input dataset to the trained autoencoder system.

It follows that the input dataset may have a plurality of data fields comprising input data corresponding to demographic data pertaining to the person and input data missing in fields which correspond to HIV and/or Syphilis status. On the other hand, the complete dataset may have demographic data, as well as HIV and Syphilis status provided in the fields. In other words, the complete dataset may have no data missing from the respective fields.

The complete dataset may comprise antenatal data comprising demographic data as well as HIV status and Syphilis status information associated with a plurality of people.

The demographic data contained in the complete dataset may comprise data selected from a group comprising race, region, age of the mother, age of the father, education level of the mother, gravidity, parity, province of origin, region of origin, and a regional weighting parameter (WTREV).

From the foregoing, it will be appreciated that the input data may also comprise demographic data selected from a group comprising race, region, age of the mother, age of the father, education level of the mother, gravidity, parity, province of origin, region of origin, and a regional weighting parameter (WTREV).

The one or more processors may be configured to normalise the antenatal data to a vector format for the complete dataset.

The one or more processors may be configured to:
prompt a person for demographic data;
receive the demographic data from the person; and
generate the input dataset for receipt by the trained autoencoder system, wherein the input dataset comprises the demographic data received from the person and has data fields pertaining to the HIV status and/or Syphilis status of the person missing.

The one or more processors may be configured to generate the input dataset by normalising the received demographic data into a predetermined format required by the trained autoencoder system. The one or more processor may therefore be configured to vectorise the demographic data received by the person.

The one or more processors may be configured to determining if the person is a female, wherein if the person is a female, the one or more processors may be configured to prompt the female person for antenatal data prior to imputing the data missing from the input dataset.

If the person is not a female, the one or more processors may be configured to determine if the male person has a female partner. If the male person has a female partner, the one or more processors may be configured to prompt the male person for antenatal data pertaining to their female partner prior to imputing the data missing from the input dataset.

The step of determining whether the person is male or female and/or if they have a female partner may be done by the one or more processor prompting the person and receiving suitable responses.

It will be appreciated by those skilled in the art that the comments above regarding the third aspect of the invention apply herein as well, mutatis mutandis. This is because the method according to the third aspect of the invention may be implemented by the system according to the fourth aspect of the invention.

According to a fifth aspect of the invention, there is provided a method for calculating an insurance premium for a person being insured, wherein the method comprising:

receiving, by a trained autoencoder system, an input dataset comprising input data which has data missing therefrom, wherein the input data is comprises demographic data associated with the person and/or data indicative of one or more health conditions associated with the person, wherein the input data set has data missing therefrom ;

processing the input data with a trained autoencoder system comprising a plurality of stacked trained autoencoders, wherein the trained autoencoder system has been trained with a plurality of complete datasets comprising complete data, wherein each complete dataset comprises complete data which comprises demographic data and one or more health conditions associated with a person;

generating an output dataset comprising output data from the trained autoencoder system based on the input dataset;

minimising an overall error function based on a relationship between the input dataset and the generated data output dataset from the trained autoencoder system to impute the data missing from the input dataset corresponding to the one or more health conditions and/or demographic data associated with the person, wherein the imputed data missing from the input dataset preserves non-linear relationships within the trained autoencoder system;

generating an output based on the imputed data missing from the input dataset corresponding to the one or more health conditions; and

using the generated output to calculate an insurance premium or contact price for the person being insured.

It will be appreciated by those skilled in the art that the comments above regarding the third aspect of the invention apply herein as well, mutatis mutandis. This is because the method according to the fifth aspect of the invention is an application of the method according to the third aspect of the invention.

It will be appreciated that in this example embodiment, the missing data could, for example, be the gender, or HIV status of the person

According to a sixth aspect of the invention, there is provided a system for calculating an insurance premium for a person being insured, wherein the system comprises:

a memory store; and

one or more processor configured to:
receive, by a trained autoencoder system, an input dataset comprising input data which has data missing therefrom, wherein the input data is comprises demographic data associated with the person and/or data indicative of one or more health conditions associated with the person, wherein the input data set has data missing therefrom;
process the input data with a trained autoencoder system comprising a plurality of stacked trained autoencoders, wherein the trained autoencoder system has been trained with a plurality of complete datasets comprising complete data, wherein each complete dataset comprises complete data which comprises demographic data and one or more health conditions associated with a person;
generate an output dataset comprising output data from the trained autoencoder system based on the input dataset;
minimise an overall error function based on a relationship between the input dataset and the generated data output dataset from the trained autoencoder system to impute the data missing from the input dataset corresponding to the one or more health conditions and/or demographic data associated with the person, wherein the imputed data missing from the input dataset preserves non-linear relationships within the trained autoencoder system;
generate an output based on the imputed data missing from the input dataset corresponding to the one or more health conditions; and
use the generated output to calculate an insurance premium or contact price for the person being insured.

It will be appreciated by those skilled in the art that the comments above regarding the fifth aspect of the invention apply herein as well, mutatis mutandis. This is because the method according to the fifth aspect of the invention may be implemented by the system according to the sixth aspect of the invention.

According to a seventh aspect of the invention, there is provided a computer readable medium containing non-transitory instructions for controlling at least one programmable automated processor to perform any of the methods and/or method steps described above.

### BRIEF DESCRIPTION OF THE DRAWINGS

FIG. 1 shows a schematic diagram of a network comprising a system in accordance with an example embodiment of the invention;

FIG. 2 shows a schematic diagram of the system of FIG. 1 in more detail;

FIG. 3 shows schematic diagram of an autoencoder in accordance with an example embodiment of the invention;

FIG. 4 shows schematic diagram of the processor or FIG. 2 in accordance with an example embodiment of the invention;

FIG. 5 shows a flow diagram of a method in accordance with an example embodiment of the invention;

FIG. 6 shows another flow diagram of a method in accordance with an example embodiment of the invention;

FIG. 7 shows yet another flow diagram of a method in accordance with an example embodiment of the invention;

FIG. 8 shows another flow diagram of a method in accordance with an example embodiment of the invention; and

FIG. 9 shows a diagrammatic representation of a machine in the example form of a computer system in which a set of instructions for causing the machine to perform any one or more of the methodologies discussed herein, may be executed.

### DETAILED DESCRIPTION OF THE DRAWINGS

The following description of the invention is provided as an enabling teaching of the invention. Those skilled in the relevant art will recognise that many changes can be made to the embodiment described, while still attaining the beneficial results of the present invention. It will also be apparent that some of the desired benefits of the present invention can be attained by selecting some of the features of the present invention without utilising other features. Accordingly, those skilled in the art will recognise that modifications and adaptations to the present invention are possible and can even be desirable in certain circumstances, and are a part of the present invention. Thus, the following description is provided as illustrative of the principles of the present invention and not a limitation thereof.

It will be appreciated that the phrase “for example,” “such as”, and variants thereof describe non-limiting embodiments of the presently disclosed subject matter. Reference in the specification to “one example embodiment”, “another example embodiment”, “some example embodiment”, or variants thereof means that a particular feature, structure or characteristic described in connection with the embodiment(s) is included in at least one embodiment of the presently disclosed subject matter. Thus, the use of the phrase “one example embodiment”, “another example embodiment”, “some example embodiment”, or variants thereof does not necessarily refer to the same embodiment(s).

Unless otherwise stated, some features of the subject matter described herein, which are, described in the context of separate embodiments for purposes of clarity, may also be provided in combination in a single embodiment. Similarly, various features of the subject matter disclosed herein which are described in the context of a single embodiment may also be provided separately or in any suitable sub-combination.

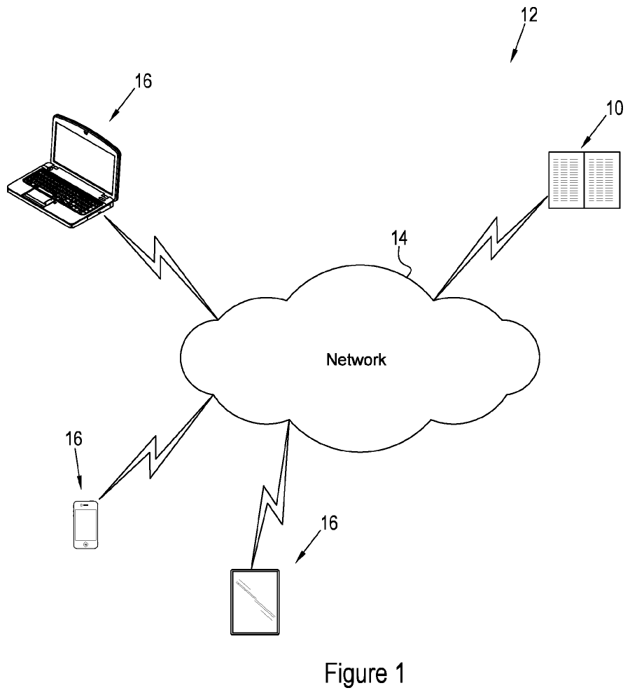

Referring to FIG. 1 of the drawings, a network comprising a system 10 in accordance with an example embodiment of the invention is generally indicated by reference numeral 10.

The system 10 is typically a computer system to impute data missing from an input dataset thereto. In numerous computational tasks in business, science, engineering, etc. the problem of missing data often arises due to inter alia sensor and system failure, non-collection, data losses, etc. Though in cases stop-gap measures such as the use of redundancies and mean imputation have been successful in determining missing data, the system 10 seeks to provides as alternative means for imputation of missing data as described herein.

For ease of explanation, and by way of a non-limiting example, the system 10 may be described with reference to an example embodiment wherein the system 10 is for determining/predicting/imputing a medical condition of a person based on input data lacking explicit information of the medical condition itself. In particular, the system 10 may be a computer system for determining/predicting an HIV and/or syphilis status of a person based on an input dataset comprising input data indicative of demographic and/or health data associated with the person and no information pertaining to the HIV and/or Syphilis status of the person, the latter being considered as missing data from the input dataset. The system 10 is thus a non-pathological computer system for determining a medical condition of a person/diagnosing a malady based on demographic and/or health data associated with a person.

It will be evident by those skilled in the art that the description which follows may be applicable to other applications of the subject matter disclosed herein.

In any event, the system 10 is typically connected to and accessible over a communications network 14 by a plurality of users via suitable endpoint computing devices 16. Though a limited number of devices 16 are shown for ease of illustration, it will be understood that the system 10 may be accessible by a plurality of users via suitable endpoint device 16. The system 16 may thus be configured to receive inputs from the devices 16 and provide suitable outputs which may be transmitted to the device 16 as well as other devices, for example, computing devices not illustrated and connectable in a hardwired fashion directly to the system 10.

The communications network 14 may comprise one or more different types of communication networks. In this regard, the communication networks may be one or more of the Internet, a local area network (LAN), a wide area network (WAN), a metropolitan area network (MAN), various types of telephone networks (e.g., Public Switch Telephone Networks (PSTN) with Digital Subscriber Line (DSL) technology) or mobile networks (e.g., Global System Mobile (GSM) communication, General Packet Radio Service (GPRS), Code Division Multiple Access (CDMA), and other suitable mobile telecommunication network technologies), or any combination thereof. It will be noted that communication within the network may achieved via suitable wireless or hard-wired communication technologies and/or standards (e.g., wireless fidelity (Wi-Fi®), 4G, long-term evolution (LTETM), WiMAX, 5G, and the like).

The endpoint computing device 16, or any computing device contemplated herein, may comprise one or more computer processors and a computer memory (including transitory computer memory and/or non-transitory computer memory), configured to perform various data processing operations. The devices 16 also include a network communication interface (not shown) to connect to the system 10 via the network 14. Examples of the devices represented by the device 16 may be selected from a group comprising a personal computer, portable computer, smartphone, tablet, notepad, dedicated server computer devices, any type of communication device, and/or other suitable computing devices. It will be appreciated that in some example embodiments, the devices 16 may be connected to network 14 via an intranet, an Internet Service Provider (ISP) and the Internet, a cellular network, and/or other suitable network communication technology.

The system 10 is typically embodied in one or more servers which are operatively communicatively connected to the network 14 by suitable network interface/s. Though one server is illustrated, it will be appreciated that the system 10 may be incorporated in one or a plurality of networked servers spread out locally and/or geographically through the network 14, for example, in a cloud-based computing like fashion.

Though not illustrated, it will be understood that the system 10 may include one or more of a back-end (e.g., a data server), a middleware (e.g., an application server), and a front-end (e.g., a client computing device having a graphical user interface (GUI) or a Web browser through which a user can interact with example implementations of the subject matter described herein). In a preferred example the example embodiment under discussion, the graphical user interface or Web browser may be rendered on the computing devices 16. In particular, the users may access the system 10 via the network 14 by entering, on a web browser, a Uniform Resource Locator (URL) corresponding to a domain hosted by the system 10. Accordingly, a web page with the GUI is displayed on computing device 16.

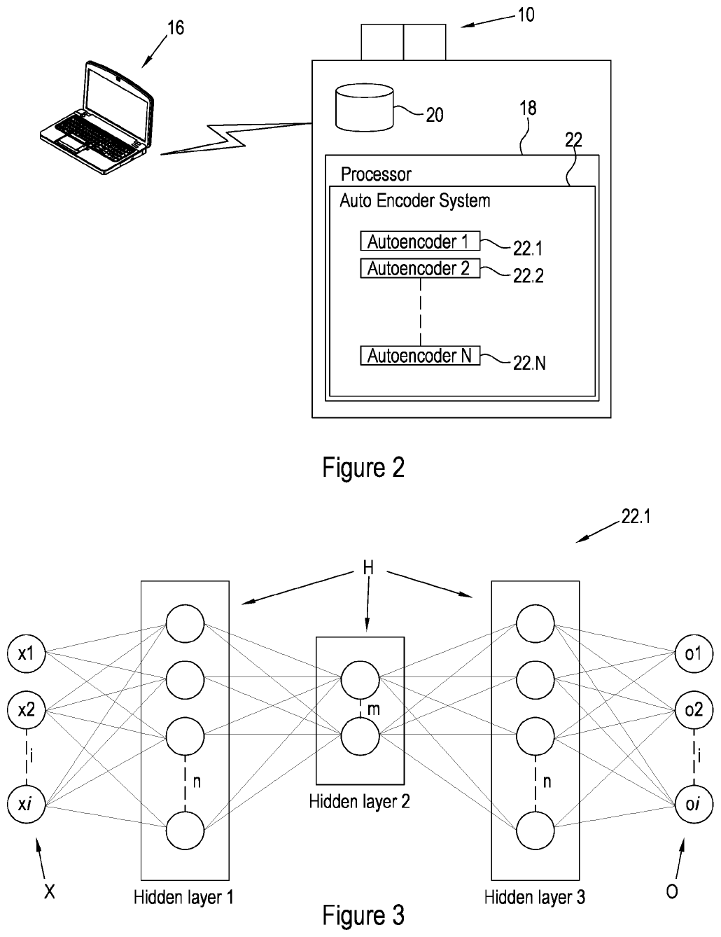

Referring now also to FIGS. 2 and 3 of the drawings, the system 10, particularly the one or more servers, may include a processor 18 and memory store or computer memories 20 (including transitory computer memory and/or non-transitory computer memory), which are configured to perform various data processing and communication operations associated with imputing missing data from an input dataset as described herein. It will be noted that the system 10 may be configured to receive the input dataset from the device 16.

The processor 18 may be one or more processors in the form of programmable processors executing one or more computer programs to perform actions by operating on input data and generating output. The processor 18, as well as any computing device referred to herein, may be any kind of electronic device with data processing capabilities including, by way of non-limiting example, a general processor, a graphics processing unit (GPU), a digital signal processor (DSP), a microcontroller, a field programmable gate array (FPGA), an application specific integrated circuit (ASIC), or any other electronic computing device comprising one or more processors of any kind, or any combination thereof. For brevity, steps described as being performed by the system 10 may be steps which are effectively performed by the processor 18 and vice versa unless otherwise indicated.

It will be appreciated that the memory store 20 may be a database. The memory store 20 may be in the form of computer-readable medium including system memory and including random access memory (RAM) devices, cache memories, non-volatile or back-up memories such as programmable or flash memories, read-only memories (ROM), etc. In addition, the memory store 20 may be considered to include memory storage physically located elsewhere in the system 10, e.g. any cache memory in the processor 18 as well as any storage capacity used as a virtual memory, e.g., as stored on a mass storage device.

Though not illustrated, it will be appreciated that the system 10 may comprise one or more user input devices (e.g., a keyboard, a mouse, imaging device, scanner, microphone) and a one or more output devices (e.g., a Liquid Crystal Display (LCD) panel, a sound playback device (speaker), switches, valves, etc.).

It will be appreciated that the computer programs executable by the processor 18 may be written in any form of programming language, including compiled or interpreted languages, declarative or procedural languages, and can be deployed in any form, including as a stand-alone program or as a module, component, subroutine, object, or other unit suitable for use in a computing environment. The computer program may, but need not, correspond to a file in a file system. The program can be stored in a portion of a file that holds other programs or data (e.g., one or more scripts stored in a mark-up language document), in a single file dedicated to the program in question, or in multiple coordinated files (e.g., files that store one or more modules, sub-programs, or portions of code). The computer program can be deployed to be executed by one processor 18 or by multiple processors 18, even those distributed across multiple locations, for example, in different servers and interconnected by the communication network 14.

The computer programs may be stored in the memory store 20 or in memory provided in the processor 18. Though not illustrated or discussed herein, it will be appreciated by those skilled in the field of invention that the system 10 may comprise a plurality of logic components, electronics, driver circuits, peripheral devices, etc. not described herein for brevity.

In any event, the processor 18 is configured/programmed to apply/provide a trained autoencoder system 22, wherein the trained autoencoder system 22 comprises a plurality of stacked autoencoders 22.1 . . . . 22.N which have been trained with one or more complete datasets.

In some example embodiments, the processor 18 may first train the autoencoder system 22 prior to use. However, in some example embodiments, the processor 18 may be configured/programmed to apply or provide a pre-trained autoencoder system 22. In this regard, the trained autoencoder system 22 may be stored in the memory store 20 and/or memory in the processor 18.

Practically, in applying/providing the autoencoder system 22, the processor 18 provides or applies a plurality of trained non-linear functions which have optimal weights which have been derived from training on complete datasets as will be described below.

It will be appreciated by those skilled in the art that unless described in the context of training the same, reference to the autoencoder system 22 or autoencoders 22.1 . . . 22.N will be reference to the trained autoencoder system 22 or autoencoders 22.1 . . . 22.N.

Referring also to FIG. 3 of the drawings where an example autoencoder 22.1 similar to each of the autoencoders 22.2 . . . 22.N, is illustrated. It follows that the explanations regarding the autoencoder 22.1 apply equally to each of the autoencoders 22.2 . . . 22.N of the autoencoder system 22.

In any event, the autoencoder 22.1 of the type illustrated is well known in prior art and is essentially a neural network that is trained to recall as outputs what they have seen as inputs. The autoencoder 22.1 has an input layer X, an output layer O, and a few hidden layers H. The autoencoder 22.1 may be constructed using various forms of neural networks such as the multi-layer perceptron, radial basis functions, deep networks, and the like. In this way, an autoencoder 22.1 operates by nonlinearly mapping the variables onto themselves.

The outputs of the autoencoder 22.1 may be defined as:

Oᵢ=f (w, X) (1)

wherein f is a function that propagates the vector of inputs through weighted non-linear functions in the hidden layers of the autoencoder 22.1. The w's are all the weights of the autoencoder 22.1.

In one example embodiment, each autoencoder 22.1 . . . 22.N of the autoencoder system 22 is trained with a plurality of complete datasets having HIV and/or Syphilis status of a person as well as demographic and/or health data associated therewith. In one example embodiment, the complete datasets may be derived from antenatal data collected as part of National Antenatal HIV Prevalence Surveys.

The antenatal date may be normalised and/or converted into a vector format for use in training of the autoencoder system 22. To this end, the complete dataset may be in the form of a vector having a predetermined number of fields/dimensions corresponding to the information contained therein comprise fields pertaining to HIV (Human Immunodeficiency Virus) status and/or Syphilis status, and multiple other fields selected from a group comprising race, gender, region, age of the mother, age of the father, education level of the mother, gravidity, parity, province of origin, region of origin, and a regional weighting parameter (WTREV). It will be appreciated that the number of fields of data determines the dimensions of the complete dataset.

The qualitative variables such as race and region are converted into integer values. The age of mother and father are represented in years. The integer value representing education level represents the highest grade successfully completed, with 13 representing tertiary education. Gravidity is the number of pregnancies, complete or incomplete, experienced by a female, and this variable is represented by an integer between 0 and 11. Parity is the number of times the individual has given birth, (for example, multiple births are counted as one) and this is not the same as gravidity.

In training the autoencoder system 22 with the complete dataset, it will be appreciated that the combination of demographic and health data as well as the HIV and syphilis status are mapped onto itself with each autoencoder 22.1 . . . 22.N.

Training each autoencoder 22.1 . . . 22.N with the complete datasets effective attempt to derive optimal weights in Equation 1 for each autoencoder 22.1 . . . 22.N. This is achieved by minimising an error function associated with a respective autoencoder.

Referring again to FIG. 3, the overall error of the autoencoder 22.1 is defined by an appropriate distance metric between the inputs X and the outputs O, for example, using Euclidean distance as shown below:

$$E ( w ) = ( \begin{matrix}{x}_{1} & {o}_{1} \\ {x}_{..} & {o}_{..} \\ {x}_{i} & {o}_{i}\end{matrix} ) \qquad ( 2 )$$

Where xᵢ's are inputs and oᵢ's are outputs which are functions of the weights w.

The error function defined by Equation 2 may be minimised by techniques such as gradient decent, Particle Swam optimisation, genetic algorithm, etc. In the case of training, it will be understood that the inputs X are complete datasets and outputs O are from the respective autoencoder 22.1 . . . 22.N being trained.

It will be noted that once the optimal weights for each autoencoder 22.1 . . . 22.N has been derived, the processor 18 stores these weights, for example, in the memory store 20. The processor 18 may be configured to train the autoencoders 22.1 . . . 22.N in parallel to obtain an output vector which is a weighted combination of the outputs of the parallel encoders 22.1 . . . 22.N.

For ease of explanation, the terms “complete dataset”, “input dataset”, and “output dataset” may be understood to be vectors comprising data and may thus be used interchangeably, unless indicated otherwise, with the terms “complete vector”, “input vector” , and “output vector”. Moreover, the term “data” with respect to the terms “vector” and “dataset” may be understood to be information contained in the fields of the dataset/vector. Thus “missing data” may be understood to mean fields which do not have data, which will be the fields pertaining to HIV and/or Syphilis status.

The processor 18 is configured to error weight each output dataset from each of the trained autoencoders 22.1 . . . 22.N to generate an error weighted output dataset from the autoencoder system 22 For brevity, the error output dataset from autoencoder system 22 is depicted by the equation below:

$${O}_{stacked} = \frac{{E}_{1}}{{E}_{a l l}} {O}_{autoencoder \_ 1} + \frac{{E}_{2}}{{E}_{a l l}} {O}_{autoencoder \_ 2} + … + \frac{{E}_{n}}{{E}_{a l l}} {O}_{autoencoder \_ n} , \qquad ( 3 )$$

where the error the N^{th} autoencoder is Eₙ and the total overall error is Eₐₗₗ

It will be appreciated that in this way, the weighting of the of the output from each of the autoencoders 22.1 . . . 22.N is dependent on the performance on the respective autoencoder during training. It therefore follows that the Equation 3 may be but one way of achieving this. For discreate imputations majority vote is also a possibility. For example, if may autoencoders 22.1 . . . . 22.N have output imputations which converge, i.e., are the same or similar, then that output imputation is selected as the output of the autoencoder system 22.

Moreover, it will be noted that the overall error above is the sum of all the errors of the individual autoencoders 22.1 . . . 22.N during training.

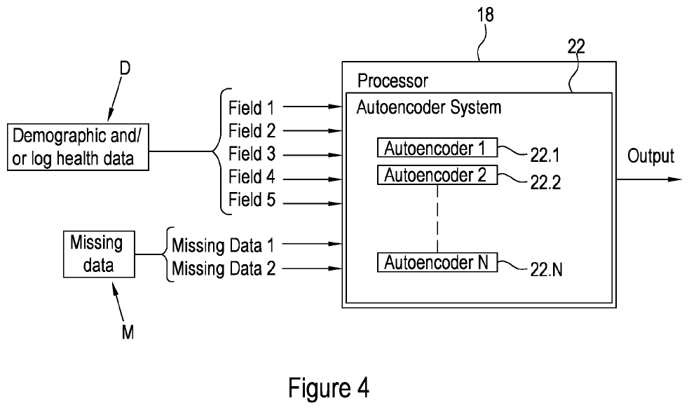

Referring also to FIG. 4 of the drawings, the processor 18 is configured to receive an input dataset of same dimensions as the complete dataset comprising input data in the form of demographic and/or health data D in the various fields of the input dataset as well as missing data M for fields which, in the case of the example embodiments under discussion, pertain to HIV and Syphilis status of a person. The input dataset may be received by the system 10 from a user by way of the device 16 over the network 14.

In some example embodiments, the processor 18 may be configured to generate the input dataset based on responses to prompts for data from users. To this end, the processor 18 may be configured to prompt users for demographic and/or health data D and purposefully omit prompting the users for the HIV and Syphilis status, the latter being the missing data M as described above. In alternate example embodiments, the missing data is the data which the user has failed to provide, for example, gender or any other demographic data.

The processor 18 may normalise the responses received, for example, by assigning integers to the demographic and/or health data D as described above. The processor 18 is further configured to generate the input dataset by populating a vector with the demographic and/or health data D, as normalised, and omitting data from fields corresponding to HIV and Syphilis status of the person.

The processor 18 is then configured to process the input dataset with the autoencoder system 22 to generate the error weighted output dataset as described above.

The processor 18 is further configured to minimise an overall error function, which is not different from that described above in Equation 2, wherein the input data X is the input dataset having data missing therefrom as described above and the output data O is the error weighted output dataset as per Equation 3 above.

The processor 18 is configured to impute the data missing from the input dataset which preserves non-linear relationships within the trained autoencoder system 12.

The processor 18 is further configured to generate an output based on the imputed data missing from the input dataset which in the example embodiment is the HIV and Syphilis status of a person. The output may be a response message, for example, to the user which transmitted the input dataset to the system 10.

In some example embodiments, the system 10 may be/may be part of/may be communicatively coupled to an insurance system (not shown), which uses the output from the processor 18, i.e., the imputed or predicted HIV and Syphilis status of a person, to calculate an insurance premium and/or a contract price for a life insurance financial product.

In example embodiments, where the system 10 is a system for calculating an insurance premium or contract price for a life insurance financial product, the processor may be configured to calculate the insurance premium and/or a contract price.

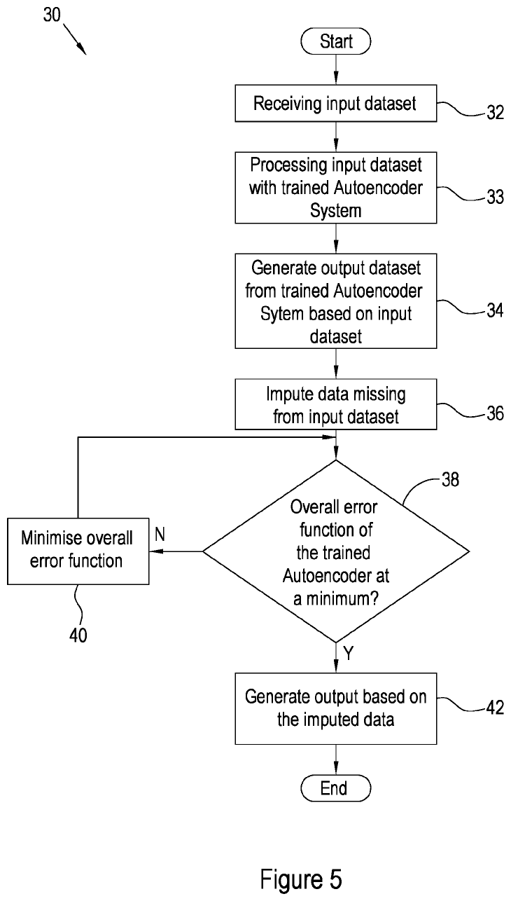

Referring now to FIGS. 5 to 8 of the drawings where flow diagrams of methods in accordance with example embodiments of the invention are generally indicated by reference numerals 30, 50, 60, and 80. It will be appreciated that the example methods 30, 50, 60, and 80 may be implemented by computer systems and means not described herein. However, by way of a non-limiting example, reference will be made to the methods 30, 50, 60, and 80 as being implemented by way of the system 10 as described above.

Referring to FIG. 5 of the drawing wherein the method 30 is a method of imputing missing data from an input dataset. In particular, the method 30 is for probabilistically determining a medical condition, viz. the HIV and syphilis status, of a person based on an input dataset which contains demographic and/or health data about the person but does not contain data pertaining to the HIV and Syphilis status (missing data from the input dataset) using autoencoders trained on complete datasets. In this regard, the method 30 essentially imputes the HIV and Syphilis status of a person based on machine learned non-linear relationships between the HIV and Syphilis status and demographic and/or health data from complete datasets which comprise not only demographic and/or health data but also data indicative of HIV and Syphilis status of people.

The method 30 comprises receiving, at block 30, an input dataset comprising input data which has data missing therefrom. In particular, the input dataset comprises data in the fields pertaining to demographic and/or health of a person and no and/or incorrect information in the fields pertaining to HIV and syphilis status of the person.

The method 30 comprises processing the input dataset, at block 33, with a trained autoencoder system, for example, system 22 comprising a plurality of stacked trained autoencoders 22.1 . . . 22.N. The trained autoencoder system 22 was previously trained with a plurality of complete datasets, each having fields with demographic and/or health of a person as well as correct/complete information in the fields pertaining to HIV and syphilis status of the person.

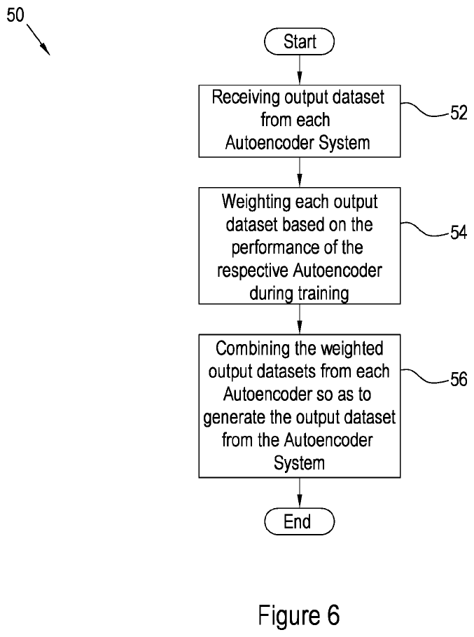

The method 30 then comprises generating, at block 34, an output dataset comprising output data from the trained autoencoder system 22. As mentioned above, the output dataset may be error weighted as per Equation 3 above, this is described below with reference to method 50 as illustrated in FIG. 6.

The method 30 may computing imputing, at block 36 by way of the processor 18, the data missing from the input dataset, i.e., the HIV and Syphilis status of a person by minimising an overall error function of the autoencoder system 22 as described above.

In particular, the method 30 may determine, at block 38, if the overall error function is at a minimum. If no, then the method 30 comprises minimising, at block 40, the overall error function until the error function is at a minimum.

The minimisation step 40 may comprise using optimisation algorithms such as genetic algorithm, particle swarm optimisation, and the like to minimise the error.

If the error function is indeed at a minimum then the method 30 may comprise generating, at block 42 by way of the processor 18, an output based on the imputed data described above.

As alluded to above, the method 30 may comprise (not shown) the step of calculating an insurance premium and/or contract price for an insurance product for a person based on the output of block 42. In this way, an insurance company is able to determine in a probabilistic way the HIV and Syphilis status of a prospective client and underwrite any insurance products accordingly. This saves costs and resources to be expended on having to have the prospective client attend pathology testing, etc.

As alluded to above, as the autoencoder system 22 is trained with a complete dataset comprising both demographic and health data, the method 30 and the system 10 described herein may be able to impute any missing/incorrect/corrupt data from an input dataset including missing demographic and/or health data.

Those skilled in the field of invention will appreciate that the imputation as described herein may be used to impute other missing/corrupt/erroneous data from input data sets using autoencoders which have been trained on associated complete datasets.

Referring to FIG. 6 of the drawings wherein the method 50, as mentioned with respect to block 34 above, is for error weighting the output data from the autoencoders 22.1 . . . 22.N.

To this end, the method 50 may comprise receiving an output dataset from each autoencoder 22.1 . . . 22.N, at block 52 by way of the processor 18.

The method 50 then comprises weighting, at block 54 by way of the processor 18, each output dataset from each autoencoder 22.1 . . . 22.N with an error weighting based on the performance of the respective autoencoder 22.1 . . . 22.N during training thereof. To this end, though not illustrated, the method 50 comprises determining an error of each autoencoder 22.1 . . . 22.N, determining an overall error of the autoencoder system 22, and obtaining the error weighting for each autoencoder 22.1 . . . 22.N by determining a ratio between the determined error of a respective autoencoder 22.1 . . . 22.N and the determined overall error of the autoencoder system 22. The last step of determining the ration may be achieved by dividing the determined error of a respective autoencoder 22.1 . . . 22.N by the determined overall error of the autoencoder system 22. The method 50 may then comprise multiplying each output of the respective autoencoder 22.1 . . . 22.N with the associated determined error weighting to obtain weighted output datasets from each autoencoder 22.1 . . . 22.N.

The method 50 may then comprise combining, at block 56 also by way of the processor 18, the weighted output datasets from each autoencoder 22.1 . . . 22.N so as to generate the output dataset from the autoencoder system 22 as described herein. It will be appreciated that this may be achieved by adding the weighted output datasets from each autoencoder 22.1 . . . 22.N as per Equation 3 described above.

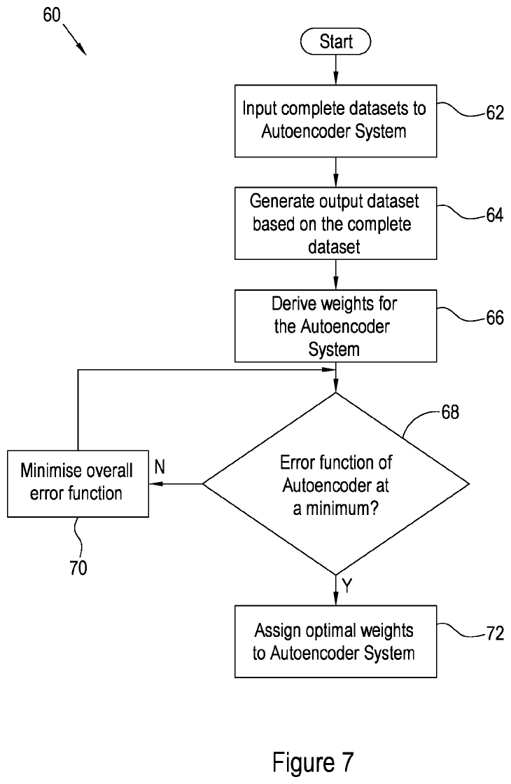

Referring to FIG. 7 of the drawings where method 60 is for training an autoencoder system, for example, the autoencoder system 22 to be able to impute the missing data as described above. It will be understood that the method 60 may be a prior step to the method 30 as it may be computationally exhaustive to be done as part of the imputation method described above. Moreover, the method 60 may be a method for training a plurality of autoencoders in parallel.

In any event, the method 60 comprises inputting complete datasets as described above, at block 62 to each autoencoder in an untrained autoencoder system.

The method 60 further comprises generating, at block 64, output datasets from each of the autoencoder based on the complete dataset.

The method 60 comprises deriving, at block 66, weights for each autoencoder. It will be appreciated that these steps may be conventional in machine learning and may effectively be deriving a weighted encoder function of each autoencoder.

The method 60 may comprise determining, at block 68, if the derived weights minimise an error function associated with the autoencoder. If not, the method 60 may comprise minimizing the error function at block 70 until optimal weights are derived. This may be achieved by using an optimisation algorithm such as genetic algorithm, particle swarm optimisation.

If the error is minimised, the method 60 comprises, at block 72, assigning the optimal weights to the respective autoencoder so as to yield a trained autoencoder 22.1 . . . 22.N.

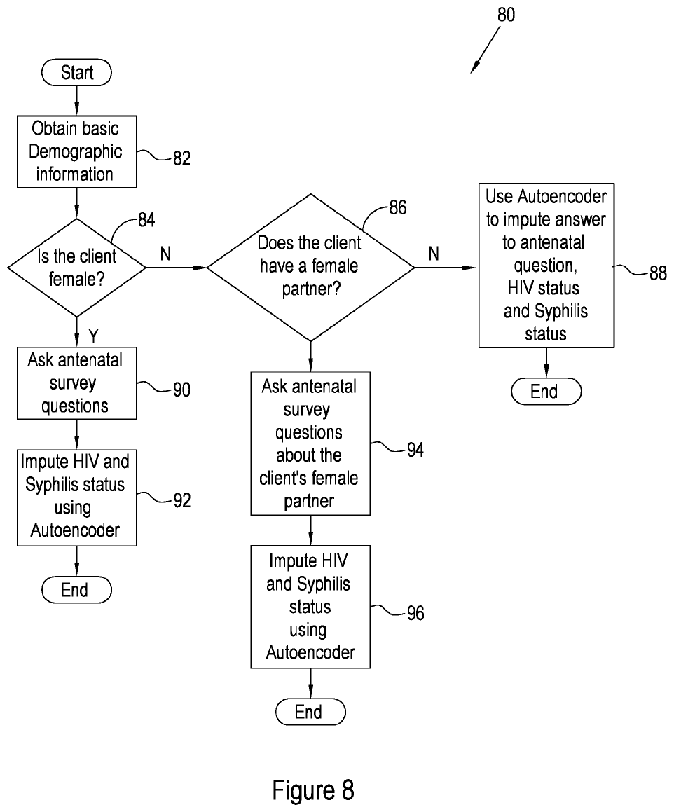

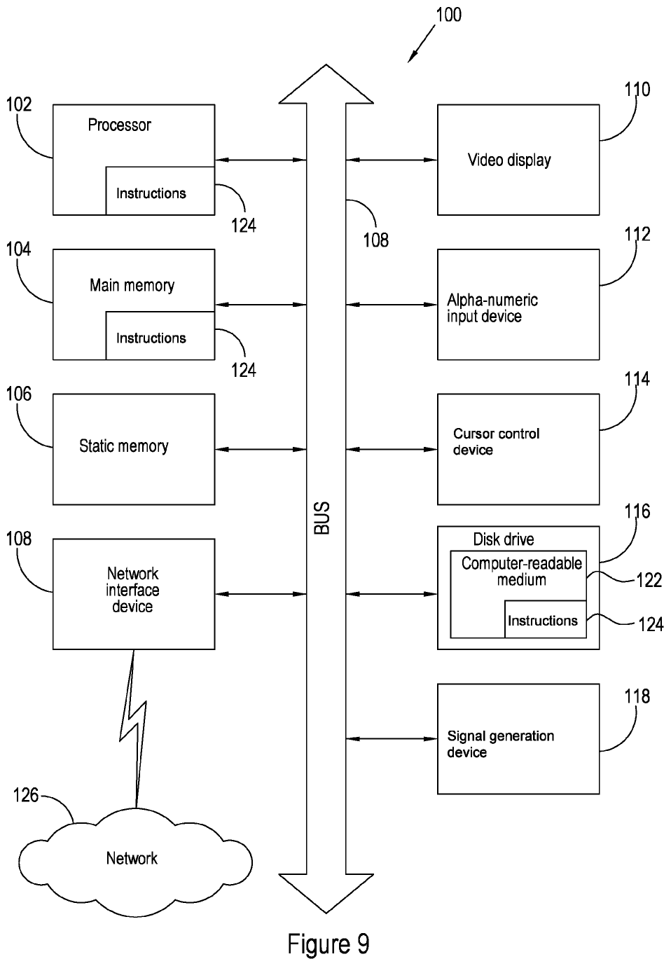

Referring to FIG. 8 of the drawings, the method 80 is typically a high-level method which illustrates an example embodiment of an application of the subject matter disclosed herein to use in an insurance system. In particular, where a new client presents themselves to an insurance contract issuer and the HIV and/or syphilis status are imputed using the system and/or method described herein. The new client may access the system 10 via the device 16 by inputting data into the device 16 which is transmitted via the network 14 to the system 10 or the client may liaise with an intermediary human operator such as a call centre agent operating the device 16 which is communicatively coupled to the system 10 to input and receive data therefrom.

The method 80 comprises receiving, at block 82 via the processor 18, basic demographic information from the client.

The method 80 comprises determining, at block 84 based on the demographic information received above or separately from the client, the gender of the client.

If the client is a male, the method 80 comprises determining, at block 86 if the male client has a female partner. This may be achieved by prompting the client for this information.

If the client does not have a female partner, the method 80 comprises imputing, at block 88, the HIV status and Syphilis status, as well as any missing demographic data in a manner as described herein by effectively classifying the missing demographic data and HIV and Syphilis status as missing data in the input dataset as described herein.

If at block 84, the client is female, the method 80 comprises prompting the client for antenatal data, at block 90, and imputing the HIV status and Syphilis status, at block 92, in a manner as described above.

If at block 86, the client has a female partner, the method 80 comprises prompting the client for antenatal data, at block 94, pertaining to the client's female partner and imputing the HIV status and Syphilis status, at block 96, in a manner as described above.

It will be understood that prompting the client for information in the method 80 may comprise the steps (not shown) of receiving the information and optionally storing it in the memory store 20, for example.

Referring now to FIG. 7 of the drawings which shows a diagrammatic representation of machine in the example of a computer system 100 within which a set of instructions, for causing the machine to perform any one or more of the methodologies discussed herein, may be executed. In other example embodiments, the machine operates as a standalone device or may be connected (e.g., networked) to other machines. In a networked example embodiment, the machine may operate in the capacity of a server or a client machine in server-client network environment, or as a peer machine in a peer-to-peer (or distributed) network environment. The machine may be a personal computer (PC), a tablet PC, a set-top box (STB), a Personal Digital Assistant (PDA), a cellular telephone, a web appliance, a network router, switch or bridge, or any machine capable of executing a set of instructions (sequential or otherwise) that specify actions to be taken by that machine. Further, while only a single machine is illustrated for convenience, the term “machine” shall also be taken to include any collection of machines, including virtual machines, that individually or jointly execute a set (or multiple sets) of instructions to perform any one or more of the methodologies discussed herein.

In any event, the example computer system 100 includes a processor 102 (e.g., a central processing unit (CPU), a graphics processing unit (GPU) or both), a main memory 104 and a static memory 106, which communicate with each other via a bus 108. The computer system 100 may further include a video display unit 110 (e.g., a liquid crystal display (LCD) or a cathode ray tube (CRT)). The computer system 100 also includes an alphanumeric input device 112 (e.g., a keyboard), a user interface (UI) navigation device 114 (e.g., a mouse, or touchpad), a disk drive unit 116, a signal generation device 118 (e.g., a speaker) and a network interface device 120.

The disk drive unit 16 includes a non-transitory machine-readable medium 122 storing one or more sets of instructions and data structures (e.g., software 124) embodying or utilised by any one or more of the methodologies or functions described herein. The software 124 may also reside, completely or at least partially, within the main memory 104 and/or within the processor 102 during execution thereof by the computer system 100, the main memory 104 and the processor 102 also constituting machine-readable media.

The software 124 may further be transmitted or received over a network 126 via the network interface device 120 utilising any one of a number of well-known transfer protocols (e.g., HTTP).

Although the machine-readable medium 122 is shown in an example embodiment to be a single medium, the term “machine-readable medium” may refer to a single medium or multiple medium (e.g., a centralized or distributed memory store, and/or associated caches and servers) that store the one or more sets of instructions. The term “machine-readable medium” may also be taken to include any medium that is capable of storing, encoding or carrying a set of instructions for execution by the machine and that cause the machine to perform any one or more of the methodologies of the present invention, or that is capable of storing, encoding or carrying data structures utilised by or associated with such a set of instructions. The term “machine-readable medium” may accordingly be taken to include, but not be limited to, solid-state memories, optical and magnetic media, and carrier wave signals.

1. A computer-implemented method for imputing data missing from an input dataset, wherein the method comprises:
receiving, by a trained autoencoder system, an input dataset comprising input data which has data missing therefrom;
processing the input data with a trained autoencoder system comprising a plurality of stacked trained autoencoders, wherein the trained autoencoder system has been trained with one or more complete datasets;
generating an output dataset comprising output data from the trained autoencoder system based on the input dataset;
minimising an overall error function based on a relationship between the input dataset and the generated data output dataset from the trained autoencoder system to impute the data missing from the input dataset which preserves non-linear relationships within the autoencoder system; and
generating an output based on the imputed data missing from the input dataset.

2. The method as claimed in claim 1, wherein the method comprises generating the output substantially in real-time.

3. The method as claimed in either claim 1 or 2, wherein the complete dataset, the input dataset, and the output dataset each have a similar data structures.

4. The method as claimed in claim 3, wherein the complete dataset, the input dataset, and the output dataset have the same dimensionality.

5. The method as claimed in claim 1, wherein the complete dataset, the input dataset, and the output dataset have the same predetermined number of fields, wherein the input dataset has one or more fields with missing data whereas the complete dataset has no missing data in the fields.

6. The method as claimed in any one of the preceding claims, wherein the method comprises training an autoencoder system comprising a plurality of stacked autoencoders with one or more complete datasets to generate the trained autoencoder system comprising a plurality of trained autoencoders.

7. The method as claimed in claim 6, wherein each autoencoder comprises a neural network, wherein the neural network comprises an input layer, at least one hidden layer, and an output layer, and wherein the input layer has the same dimensionality as the output layer.

8. The method as claimed in either claim 6 or 7, wherein the training of the autoencoder system comprises, for each autoencoder in the autoencoder system:
inputting one or more complete datasets into an autoencoder;
generating an output dataset which is outputted from the autoencoder based on the complete dataset; and
deriving optimal weights for the respective autoencoder or the weighted encoder function of the autoencoder by minimising an error function associated with the autoencoder to yield a trained autoencoder.

9. The method as claimed in any one of claims 6 to 8, wherein the method comprises training each of the plurality of autoencoders in the autoencoder system in parallel to derive the trained autoencoder system.

10. The method as claimed in claim 8, wherein the trained autoencoder system comprises trained autoencoders having the derived optimal weights assigned thereto.

11. The method as claimed in claim 8, wherein the error function of each autoencoder is a distance metric between the complete dataset inputted to the autoencoder and the output dataset from the autoencoder.

12. The method as claimed in claim 8, wherein the step of minimising the overall error function to impute the data missing from the input dataset comprises minimising the overall error function for the trained autoencoder system with the optimal weights associated with each autoencoder of the autoencoder system fixed.

13. The method as claimed in claim 6, wherein the step of determining an output dataset from the trained autoencoder system comprises combining products of the output datasets of each trained autoencoder and an error ratio associated with the respective trained autoencoders, wherein the error ratio is based on an error of a particular autoencoder and an overall error of the autoencoder system.

14. The method as claimed in claim 13, wherein the overall error function of the trained autoencoder system is a distance metric between the input dataset inputted to the trained autoencoder system and the output dataset from the trained autoencoder system.

15. The method as claimed in any one of the preceding claims, wherein the complete dataset is in the form of complete antenatal data, wherein in addition to fields pertaining to HIV (Human Immunodeficiency Virus) status and/or Syphilis status, the fields are selected from a group comprising race, region, age of the mother, age of the father, education level of the mother, gravidity, parity, geographical location of origin, geographical region of origin, and a geographical regional weighting parameter (WTREV).

16. The method as claimed in claim 15, wherein the method comprises normalising the antenatal data to a vector format, wherein dimensions of input and output layers of each autoencoder in the autoencoder system are based on the number of fields selected.

17. The method as claimed in any one of the preceding claims, wherein the input dataset may be missing fields pertaining to one or both of HIV and Syphilis status of a person, wherein the method comprises imputing one or both of HIV and Syphilis status from the input dataset.

18. A computer system for imputing data missing from an input dataset, wherein the system comprises:
a data storage device storing data; and
one or more processors coupled to the data storage device and configured to:
receive an input dataset comprising input data which has data missing therefrom;
process the input data with a trained autoencoder system wherein the trained autoencoder system comprises a plurality of stacked trained autoencoders, wherein the trained autoencoder system has been trained with one or more complete datasets;
generate an output dataset comprising output data from the trained autoencoder system based on the input dataset;
minimise an overall error function based on a relationship between the input dataset and the generated data output dataset from the trained autoencoder system to impute the data missing from the input dataset which preserves non-linear relationships within the trained autoencoder system; and
generate an output based on the imputed data missing from the input dataset.

19. The system as claimed in claim 18, wherein the one or more processors are configured to provide the trained autoencoder system.

20. The system as claimed in either claim 18 or 19, wherein the one or more processors are configured to train an autoencoder system comprising a plurality of stacked autoencoders with one or more complete datasets to generate the trained autoencoder system comprising a plurality of trained autoencoders;

21. The system as claimed in claim 20, wherein the one or more processors are configured to train the autoencoder system by:
inputting, to each autoencoder in the autoencoder system, one or more complete datasets;
generating an output dataset which is outputted from each autoencoder based on the complete dataset; and
deriving optimal weights for the respective autoencoder or the weighted encoder function of the autoencoder by minimising an error function associated with the autoencoder so as to yield a trained autoencoder.

22. The system as claimed in any one of claims 19 to 21, wherein the one or more processors are configured to train each of the plurality of autoencoders in the autoencoder system in parallel to derive the trained autoencoder system.

23. The system as claimed in claim 21, wherein the trained autoencoder system comprises trained autoencoders having the derived optimal weights assigned thereto.

24. The system as claimed in claim 21, wherein the one or more processors are configured to minimise the overall error function for the trained autoencoder system with the optimal weights associated with each autoencoder of the autoencoder system fixed.

25. The system as claimed in claim 20, wherein the one or more processors are configured to determine an output dataset from the trained autoencoder system by combining products of the output datasets of each trained autoencoder and an error ratio associated with the respective trained autoencoders.

26. The system as claimed in claim 25, wherein the error ratio is based on an error of a particular autoencoder and an overall error of the autoencoder system.

27. The system as claimed in claim 26, wherein The overall error function may be a square of the difference between the input dataset and the output dataset from the trained autoencoder system.

28. A computer-implemented method of determining a health condition of a person, wherein the method comprises:
receiving, by a trained autoencoder system, an input dataset comprising input data which has data missing therefrom, wherein the input data is comprises demographic data associated with the person and the data missing from the input data is one or more health conditions associated with the person;
processing the input data with a trained autoencoder system comprising a plurality of stacked trained autoencoders, wherein the trained autoencoder system has been trained with a plurality of complete datasets comprising complete data, wherein each complete dataset comprises complete data which comprises demographic data and one or more health conditions associated with a person;
generating an output dataset comprising output data from the trained autoencoder system based on the input dataset;
minimising an overall error function based on a relationship between the input dataset and the generated data output dataset from the trained autoencoder system to impute the data missing from the input dataset corresponding to the one or more health conditions associated with the person, wherein the imputed data missing from the input dataset preserves non-linear relationships within the trained autoencoder system; and
generating an output based on the imputed data missing from the input dataset corresponding to the one or more health conditions.

29. The method as claimed in claim 28, wherein the health condition is a predictive diagnosis of a malady.

30. The method as claimed in either claim 28 or 29, wherein the health condition is a positive or negative predictive diagnosis of a person having HIV (Human Immunodeficiency Virus) and/or Syphilis based on the input dataset to the trained autoencoder system.

31. The method as claimed in claim 30, wherein the input dataset has a plurality of data fields comprising input data corresponding to demographic data pertaining to the person and input data missing in fields which correspond to HIV and/or Syphilis status.

32. The method as claimed in claim 31, wherein the demographic data contained in the complete dataset comprises data selected from a group comprising race, region, age of the mother, age of the father, education level of the mother, gravidity, parity, geographical location or province of origin, geographical region of origin, and a geographical regional weighting parameter (WTREV).

33. The method as claimed in either claim 31 or 32, wherein the input data comprises demographic data selected from a group comprising race, region, age of the mother, age of the father, education level of the mother, gravidity, parity, geographical location or province of origin, geographical region of origin, and a geographical regional weighting parameter (WTREV).

34. The method as claimed in any one of claims 28 to 33, wherein the method comprises normalising the antenatal data to a vector format for the complete dataset.

35. The method as claimed in any one of claims 28 to 34, wherein the method comprises the prior steps of:
prompting a person for demographic data;
receiving the demographic data from the person; and
generating the input dataset for receipt by the trained autoencoder system, wherein the input dataset comprises the demographic data received from the person and has data fields pertaining to the HIV status and/or Syphilis status of the person missing.

36. The method as claimed in claim 35, wherein the step of generating the input dataset comprises normalising the received demographic data into a predetermined format required by the trained autoencoder system.

37. The method as claimed in any one of claims 28 to 35, wherein the method comprises a step of determining if the person is a female, wherein if the person is a female, the method comprises prompting the female person for antenatal data prior to imputing the data missing from the input dataset.

38. The method as claimed in claim 37, wherein if the person is not a female, the method comprises determining if the male person has a female partner, wherein if the male person has a female partner, the method comprises prompting the male person for antenatal data pertaining to their female partner prior to imputing the data missing from the input dataset.

39. A system for determining a health condition of a person, wherein the system comprises:
a memory store; and
one or more processors communicatively coupled to the memory store and configured to:
receive, by a trained autoencoder system, an input dataset comprising input data which has data missing therefrom, wherein the input data is comprises demographic data associated with the person and the data missing from the input data is one or more health conditions associated with the person;
process the input data with a trained autoencoder system comprising a plurality of stacked trained autoencoders, wherein the trained autoencoder system has been trained with a plurality of complete datasets comprising complete data, wherein each complete dataset comprises complete data which comprises demographic data and one or more health conditions associated with a person;
generate an output dataset comprising output data from the trained autoencoder system based on the input dataset;
minimise an overall error function based on a relationship between the input dataset and the generated data output dataset from the trained autoencoder system to impute the data missing from the input dataset corresponding to the one or more health conditions associated with the person, wherein the imputed data missing from the input dataset preserves non-linear relationships within the trained autoencoder system; and
generate an output based on the imputed data missing from the input dataset corresponding to the one or more health conditions.

40. The system as claimed in claim 39, wherein the processor provides the trained autoencoder system.

41. The system as claimed in either claim 39 or 40, wherein the health condition is a predictive diagnosis of a malady.

42. The system as claimed in any one of claims 39 to 41, wherein the health condition is a positive or negative predictive diagnosis of a person having HIV (Human Immunodeficiency Virus) and/or Syphilis based on the input dataset to the trained autoencoder system.

43. The system as claimed in claim 42, wherein the input dataset has a plurality of data fields comprising input data corresponding to demographic data pertaining to the person and input data missing in fields which correspond to HIV and/or Syphilis status.

44. The system as claimed in claim 43, wherein the complete dataset comprises antenatal data comprising demographic data as well as HIV status and Syphilis status information associated with a plurality of people.

45. The system as claimed in claim 44, wherein the demographic data contained in the complete dataset comprises data selected from a group comprising race, region, age of the mother, age of the father, education level of the mother, gravidity, parity, geographical location or province of origin, geographical region of origin, and a geographical regional weighting parameter (WTREV).

46. The system as claimed in either claim 44 or 45, wherein the input data comprises demographic data selected from a group comprising race, region, age of the mother, age of the father, education level of the mother, gravidity, parity, geographical location or province of origin, geographical region of origin, and a geographical regional weighting parameter (WTREV).

47. The system as claimed in any one of claims 39 to 46, wherein the one or more processors are configured to:
prompt a person for demographic data;
receive the demographic data from the person; and
generate the input dataset for receipt by the trained autoencoder system, wherein the input dataset comprises the demographic data received from the person and has data fields pertaining to the HIV status and/or Syphilis status of the person missing.

48. The system as claimed in claim 47, wherein the one or more processors are configured to generate the input dataset by normalising the received demographic data into a predetermined format required by the trained autoencoder system.

49. The system as claimed in any one of claims 39 to 48, wherein the one or more processors are configured to determine if the person is a female, wherein if the person is a female, the one or more processors are configured to prompt the female person for antenatal data prior to imputing the data missing from the input dataset.

50. The system as claimed in claim 49, wherein if the person is not a female, the one or more processors are configured to determine if the male person has a female partner, wherein if the male person has a female partner, the one or more processors are configured to prompt the male person for antenatal data pertaining to their female partner prior to imputing the data missing from the input dataset.

51. A computer-implemented method for calculating an insurance premium for a person being insured, wherein the method comprising:
receiving, by a trained autoencoder system, an input dataset comprising input data which has data missing therefrom, wherein the input data is comprises demographic data associated with the person and/or data indicative of one or more health conditions associated with the person, wherein the input data set has data missing therefrom ;
processing the input data with a trained autoencoder system comprising a plurality of stacked trained autoencoders, wherein the trained autoencoder system has been trained with a plurality of complete datasets comprising complete data, wherein each complete dataset comprises complete data which comprises demographic data and one or more health conditions associated with a person;
generating an output dataset comprising output data from the trained autoencoder system based on the input dataset;
minimising an overall error function based on a relationship between the input dataset and the generated data output dataset from the trained autoencoder system to impute the data missing from the input dataset corresponding to the one or more health conditions and/or demographic data associated with the person, wherein the imputed data missing from the input dataset preserves non-linear relationships within the trained autoencoder system;
generating an output based on the imputed data missing from the input dataset corresponding to the one or more health conditions; and
using the generated output to calculate an insurance premium or contact price for the person being insured.

52. A system for calculating an insurance premium for a person being insured, wherein the system comprises:
a memory store; and
one or more processor communicatively coupled to the memory store and configured to:
receive, by a trained autoencoder system, an input dataset comprising input data which has data missing therefrom, wherein the input data is comprises demographic data associated with the person and/or data indicative of one or more health conditions associated with the person, wherein the input data set has data missing therefrom;
process the input data with a trained autoencoder system comprising a plurality of stacked trained autoencoders, wherein the trained autoencoder system has been trained with a plurality of complete datasets comprising complete data, wherein each complete dataset comprises complete data which comprises demographic data and one or more health conditions associated with a person;
generate an output dataset comprising output data from the trained autoencoder system based on the input dataset;
minimise an overall error function based on a relationship between the input dataset and the generated data output dataset from the trained autoencoder system to impute the data missing from the input dataset corresponding to the one or more health conditions and/or demographic data associated with the person, wherein the imputed data missing from the input dataset preserves non-linear relationships within the trained autoencoder system;
generate an output based on the imputed data missing from the input dataset corresponding to the one or more health conditions; and
use the generated output to calculate an insurance premium or contact price for the person being insured.

53. A non-transitory computer readable medium containing non-transitory instructions for controlling at least one programmable automated processor to perform the method as claimed in any one of claim 1 to 17, 28 to 38, or 51.
